(19) United States
(12) Patent Application Publication
(10) Pub. No.: US 2023/0320675 A1
(43) Pub. Date: Oct. 12, 2023

(54) IDENTIFICATION AND DISPLAY OF AIRWAY COLLAPSE

(71) Applicant: Covidien LP, Mansfield, MA (US)

(72) Inventor: Jeffrey P. MANSFIELD, Bloomington, IN (US)

(73) Assignee: Covidien LP, Mansfield, MA (US)

(21) Appl. No.: 18/194,771

(22) Filed: Apr. 3, 2023

Related U.S. Application Data
(60) Provisional application No. 63/329,119, filed on Apr. 8, 2022.

(51) Int. Cl. A61B 5/00; A61M 16/00; A61B 7/00

(52) U.S. Cl. CPC A61B 5/746; A61M 16/024; A61B 7/003; A61M 2230/42; A61M 2205/3375

(57) ABSTRACT

Systems and methods for identification and display of airway collapse due to conditions such as tracheomalacia (TM) or dynamic airway collapse (DAC). In an aspect, the technology relates to a method for identifying airway collapse. The method includes emitting, from an acoustic sensor, a series of acoustic pulses into a tracheal tube positioned in an airway of a patient; detecting, by the acoustic sensor, echoes resulting from the series of acoustic pulses; generating, based on the detected echoes, a time series of passageway sizes of the airway; based on the time series of passageway sizes, detecting an airway collapse has occurred; and based on detecting the airway collapse has occurred, activating an airway collapse alarm.

### CROSS-REFERENCE TO RELATED APPLICATIONS

This application claims the benefit of U.S. Provisional Application No. 63/329,119 filed Apr. 8, 2022, titled “Identification and Display of Airway Collapse,” which is incorporated herein by reference in its entirety.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. During ventilation, some patients may be connected to the ventilator through a tracheal tube that is positioned in the patient's trachea. Such tracheal tubes may include endotracheal tubes (“ETTs”), tracheotomy tubes, or transtracheal tubes. For example, a patient may be intubated when a first end of an endotracheal tube is inserted through the patient's mouth or nose and further inserted into the trachea. Then, a medical provider may couple a ventilator to a second end of the endotracheal tube and utilize the ventilator to mechanically control the type and amount of gases flowing into and out of the patient's airway. During normal conditions, the patient's trachea remains wider than that of the end or tip of the ETT. In some events, however, the patient's trachea may collapse to a diameter that is less than that that of the tip of the ETT, which may limit airflow into and out of the patient airway.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Among other things, aspects of the present disclosure include systems and methods for identification and display of airway collapse due to conditions such as tracheomalacia (TM) or dynamic airway collapse (DAC). In an aspect, the technology relates to a method for identifying airway collapse. The method includes emitting, from an acoustic sensor, a series of acoustic pulses into a tracheal tube positioned in an airway of a patient; detecting, by the acoustic sensor, echoes resulting from the series of acoustic pulses; generating, based on the detected echoes, a time series of passageway sizes of the airway; based on the time series of passageway sizes, detecting an airway collapse has occurred; and based on detecting the airway collapse has occurred, activating an airway collapse alarm.

In an example, the time series of passageway sizes are displayed on a monitor communicatively coupled to the acoustic sensor. In another example, the method further includes generating dynamic characteristics of the airway collapse based on the time series of passageway sizes. In a further example, the dynamic characteristics include at least one of a peak-to-baseline value, a baseline shift, or a baseline trend. In yet another example, the method further includes generating an airway-compliance indicator based on at least the peak-to-baseline value. In still another example, the method further includes displaying one or more of the dynamic characteristics on at least one of a monitor communicatively coupled to the acoustic sensor or a ventilator communicatively coupled to the acoustic sensor. In yet a further example, the method further includes adjusting ventilation delivered to the patient based on one or more of the dynamic characteristics. In still yet another example, the method further includes storing the time series of passageway sizes in a buffer; and based on detection of the airway collapse, storing the time series of passageway sizes in permanent memory.

In another aspect, the technology relates to a system for detecting airway collapse. The system includes an acoustic sensor comprising an acoustic receiver and an acoustic generator; a monitor communicatively coupled to the acoustic sensor; a processor; and memory storing instructions that, when executed by the processor, cause the system to perform a set of operations. The operations include emitting, from the acoustic generator, a series of acoustic pulses into a tracheal tube positioned in an airway of a patient; detecting, by the acoustic receiver, echoes resulting from the series of acoustic pulses reflecting from a tip of the tracheal tube; generating, based on the detected echoes, a time series of passageway sizes of the airway; based on at least one of the time series of passageway sizes or the detected echoes, detecting an airway collapse has occurred; and displaying the time series of passageway size on the monitor.

In an example, the system further comprises a ventilator, and the monitor is integrated into the ventilator or is a stand-alone monitor separate from the ventilator. In another example, the operations further comprise generating dynamic characteristics of the airway collapse based on the time series of passageway sizes, wherein the dynamic characteristics include at least one of a peak-to-baseline value, a baseline shift, or a baseline trend. In yet another example, the operations further comprise, based on at least one of the dynamic characteristics, adjusting, by the ventilator, a pressure of delivered breathing gases to the patient. In still another example, the operations further comprise, based on detecting the airway collapse has occurred, activating an airway-collapse alarm on the monitor.

In another aspect, the technology relates to a system for identifying an airway obstruction event. The system includes a ventilator; a monitor; an acoustic sensor communicatively coupled to at least one of the monitor or the ventilator, the acoustic sensor comprising an acoustic receiver and an acoustic generator; a processor; and memory storing instructions that, when executed by the processor, cause the system to perform a set of operations. The operations include emitting, from the acoustic generator, a series of acoustic pulses into a tracheal tube positioned in an airway of a patient; detecting, by the acoustic receiver, echoes resulting from the series of acoustic pulses reflecting from a tip of the tracheal tube; generating, based on the detected echoes, a time series of passageway sizes of the airway; based on at least one of the time series of passageway sizes or the detected echoes, detecting an airway obstruction event has occurred; generating dynamic characteristics for the time series of passageway sizes of the airway; and based on the dynamic characteristics, classifying the airway obstruction event as an airway collapse.

In an example, the dynamic characteristics include at least one of a peak-to-baseline value, a baseline shift, or a baseline trend. In another example, the dynamic characteristics include a peak-to-baseline value, wherein the peak-to-baseline value is calculated based on (1) a peak passageway size at end-inspiration of a breath delivered by the ventilator and (2) a baseline passageway size at end-expiration of a breath delivered by the ventilator. In yet another example, the operations further comprise, based on detecting the airway obstruction event, adjusting, by the ventilator, ventilation being delivered to the patient. In still yet another example, adjusting the ventilation includes increasing a positive end-expiratory pressure (PEEP) setting. In a further example, adjusting the ventilation is further based on the dynamic characteristics. In still a further example, the operations further include based on the time series of passageway sizes, detecting an end to the airway obstruction event; and downgrading an oxygenation alarm based on detecting the end to the airway obstruction event.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts an example of an airway management system.

FIG. 2 depicts an example echo signal plot.

FIG. 3A depicts a pressure plot of gas pressure versus time for a normal ventilation period where no airway collapse is occurring

FIG. 3B depicts a passageway size plot of passageway size versus time for the same normal ventilation period of FIG. 3A.

FIG. 4A depicts a pressure plot of gas pressure versus time for a time period where an airway collapse is occurring.

FIG. 4B depicts a passageway size plot of passageway size versus time for the same collapsed-airway ventilation period of FIG. 4A.

FIG. 5 depicts an example method for detecting and/or identifying an airway collapse.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently, and some patients may need to be connected to the ventilator via a tracheal tube. Such tracheal tubes may include endotracheal tubes (“ETTs”), tracheotomy tubes, or transtracheal tubes. For example, a patient may be intubated when a first end of an endotracheal tube is inserted through the patient's mouth or nose and further inserted into the trachea. Then, a medical provider may couple a ventilator to a second end of the endotracheal tube and utilize the ventilator to mechanically control the type and amount of gases flowing into and out of the patient's airway. During normal conditions, the patient's trachea remains wider than that of the end or tip of the ETT. When the patient experiences a tracheal collapse, however, the patient's trachea becomes narrower than the tip of the ETT. In the worst-case scenarios, the tracheal collapse may limit or prevent air flow into and out of the patient's lungs, which can reduce oxygenation levels of the patient (e.g., a reduction in oxygen saturation (SpO2)).

The present technology is able to identify and/or detect the occurrence of airway collapse, such as tracheal collapse, using acoustic pulses emitted through the ETT and detecting the echoes or reflections of those acoustic pulses. The echoes of the acoustic pulses are analyzed to estimate the airway passageway size (e.g., trachea cross-sectional area, trachea diameter) around the tip of the tracheal tube, and a time series of passageway sizes may be generated to create a signal that shows the changes in airway size over time. Accordingly, a collapse of the airway may be identified from the time series of passageway sizes or, in some examples, directly from the polarity and amplitude of the detected echoes themselves.

In addition, dynamic characteristics of the time series of passageway sizes may be generated to characterize the tracheal collapse. For instance, the dynamic characteristics may be indicative of physical properties of the collapse, such as the compliance of the trachea in the collapsed state. The dynamic characteristics may also be used to distinguish a collapsed trachea from other obstructive events that may cause the opening of the trachea to appear smaller, such as a mucus plug in the trachea. Based on the detection of the tracheal collapse and/or the dynamic characteristics associated therewith, the ventilation being delivered to the patient may be adjusted. The adjustment may be a temporary adjustment in an attempt to reopen the trachea, such as by temporarily increasing the pressure of the breathing gases delivered to the patient.

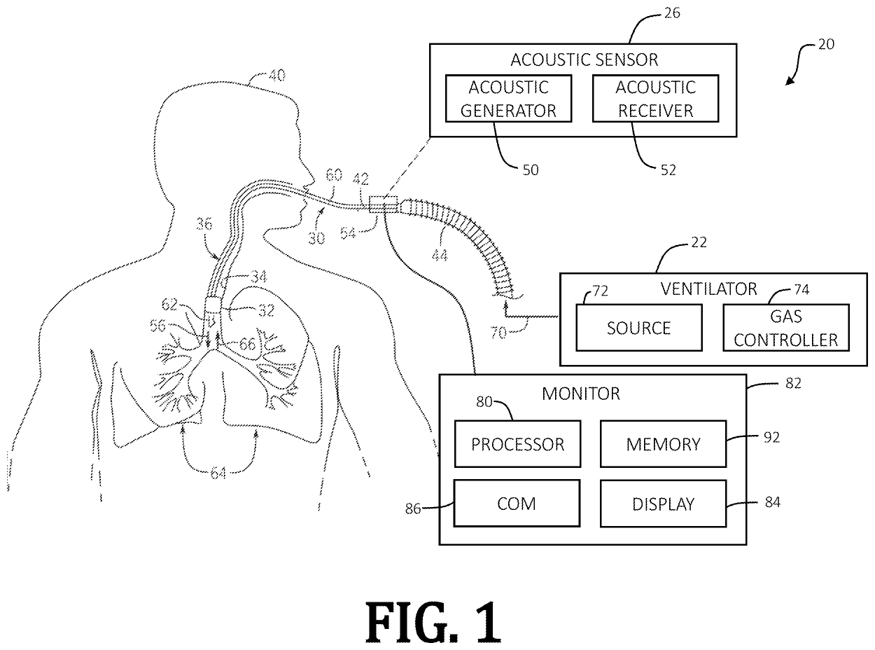

FIG. 1 depicts an example of an airway management system 20 that includes a ventilator 22, a monitor 24, an acoustic sensor 26, and a tracheal tube 30. The tracheal tube 30 is presently illustrated as an endotracheal tube, which has an inflatable balloon cuff 32 that may be inflated to form a seal against walls 34 of a trachea 36 of a patient 40. However, the tracheal tube 30 may alternatively be uncuffed. Moreover, it should be understood that the airway management system 20 may be used in conjunction with any other suitable types of tracheal tubes or medical devices. As examples, the airway management system 20 may be utilized with an endotracheal tube, an endobronchial tube, a tracheostomy tube, an introducer, an endoscope, a bougie, a circuit, an airway accessory, a connector, an adapter, a filter, a humidifier, a nebulizer, nasal cannula, or a supraglottic mask/tube.

As illustrated, the acoustic sensor 26 of the airway management system 20 is coupled to an external or proximal end 42 of the tracheal tube 30. In the illustrated example, the acoustic sensor 26 may operate as or be an adapter that facilitates coupling of the tracheal tube 30 to a patient circuit 44 or hose of the ventilator 22. Other arrangements are also contemplated, such as an acoustic sensor 26 that is disposed on the tracheal tube 30 or on other components of the breathing circuit. In some examples, the acoustic sensor 26 may be integrated directly into the ventilator 22 itself. The acoustic sensor 26 includes at least one acoustic generator 50 and at least one acoustic receiver 52 disposed within an adapter housing 54. The acoustic generator 50 is oriented to direct incident sound energy 56 (e.g., acoustic pulses, sound, acoustic energy) into a body 60 of the tracheal tube 30, which guides the sound energy 56 out of an internal or distal end 62 of the tracheal tube 30 and toward airways of lungs 64 of the patient 40. Further, the acoustic receiver 52 detects any reflected sound energy 66, or echoes of the sound energy 56, back from different positions along the endotracheal tube/and or the airways of the patient. For instance, the emitted acoustic pulses may reflect from items such as obstructions in the tracheal tube, the tip of the tracheal tube, and the airways of the lungs 64. Accordingly, the acoustic sensor 26 facilitates acoustic reflectometry techniques that analyze sound pressure waveforms for airway acoustic echoes indicative of airway size. That is, the acoustic generator 50 and the acoustic receiver 52 cooperate to provide sensor signals indicative of a sound pressure waveform having an airway acoustic echo, which the monitor 24 may analyze to determine an airway size of the trachea around, or distally located from, the tip of the tracheal tube.

The acoustic generator 50 in some examples is a speaker or a miniature speaker. However, the acoustic generator 50 may additionally or alternatively include any suitable loudspeakers, buzzers, horns, sounders, and so forth that rely on moving coil, electrostatic, isodynamic, or piezo-electric techniques. Additionally, the acoustic receiver 52 may be a microphone, microphone array, or other sound pressure sensors, in some embodiments. When implemented as a microphone array, the acoustic receiver 52 and/or the monitor 24 discussed below may be designed to sense the direction from which sound energy is emitted and received and therefore isolate or filter out any interfering sound energy that is not a reflection of the emitted sound energy 56 provided by the acoustic generator 50. Moreover, it should be understood that any other suitably paired components that respectively generate suitable sound energy and receive echoes or reflection of the sound energy may be used in the acoustic sensor 26. Additional details of a suitable acoustic sensor 26 and a monitor 24 are described in U.S. Pat. No. 9,707,363, titled “System and Method for Use of Acoustic Reflectometry Information in Ventilation Devices,” U.S. Pat. No. 10,668,240, titled “Acoustical Guidance and Monitoring System,” which are incorporated herein by reference in their entireties.

The system 20 also includes devices that facilitate positive pressure ventilation of the patient 40, such as the ventilator 22, which may include any ventilator that provides mechanical ventilation to the patient 40. For example, the ventilator 22 may provide a gas mixture 70 (e.g., from a source 72 of the gas mixture 70) through the acoustic sensor 26, through the tracheal tube 30, and to lungs 64 of the patient 40, thereby mechanically actuating rest, inspiration, and expiration phases of breathing cycles of the patient 40. In some examples, the ventilator 22 includes a gas mixture controller 74 that provides control instructions to cause the ventilator 22 to continuously or intermittently adjust a pressure and/or a composition of the gas mixture 70 provided from the source 72 and to the patient 40. For example, the gas mixture controller 74 may cause the ventilator 22 to direct air, oxygen, or another suitable gas mixture from the source 72 to the lungs 64 of the patient 40.

In the illustrated example, the monitor 24 is communicatively coupled to the acoustic sensor 26 and the ventilator 22. The monitor 24 may analyze the sensor signals from the acoustic sensor 26 via acoustic reflectometry techniques to monitor and/or determine characteristics of the airways of the patient 40, such as a size of a trachea near the tip of the tracheal tube 30. Thus, as discussed below, the monitor 24 may provide detection and/or identification of tracheal collapse in the patient 40.

In some examples, the monitor 24 may also provide control instructions to the ventilator 22 (e.g., automatically) to instruct the ventilator 22 to perform operations that assist in the detection and/or identification of tracheal collapse. In further examples, the monitor 24 may also provide control instructions to the ventilator 22 to instruct the ventilator 22 to perform operations in response to the detection or identification of the tracheal collapse. For instance, in response to detecting the tracheal collapse, the ventilator 22 may increase pressure of the delivered breathing gases in an attempt to expand the trachea or reverse the tracheal collapse. In other examples, the monitor 24 does not electronically communicate with the ventilator 22.

The monitor 24 may be configured to provide indications of airway collapse and/or airway size, such as an audio, visual, or other indication, and/or may be configured to communicate the information to another device. In an example, based at least in part upon the received signals from the acoustic sensor 26, a processor 80 of the monitor 24 may determine airway collapse and related characteristics using various algorithms disclosed herein. In general, such algorithms are stored in non-transitory computer media (e.g., memory) and executed by the processing circuitry as described below. Further, in examples of the airway management system 20 in which the acoustic receiver 52 includes a microphone array, the monitor 24 may filter out signals associated with the ventilator 22 or other sounds or vibrations emitted from outside the body of the patient 40.

It should be understood that the monitor 24 may be a stand-alone device or may be integrated into a single device with another medical device, such as the ventilator 22. Coupled to or disposed within a monitor housing 82, the monitor 24 may include a display 84, at least one communication component 86 (e.g., input/output ports, communication circuitry), user-selectable buttons, a memory 92, and processing circuitry, such as the processor 80, that are all communicatively coupled to one another to facilitate the present techniques. The medical provider may provide inputs to the monitor 24 via the user-selectable buttons and/or via a sensor (e.g., a capacitive touch screen sensor on the display 84, or other mechanical or capacitive buttons or keys on the monitor housing 82). The processor 80 may include one or more microprocessors, one or more application specific integrated circuits (ASICs), one or more general purpose processors, one or more controllers, one or more programmable circuits, or any combination thereof. For example, the processor 80 of the monitor 24 may also include or refer to control circuitry for the display 84. The memory 92 may include volatile memory, such as random-access memory (RAM), and/or non-volatile memory, such as read-only memory (ROM). Moreover, the processor 80 may execute instructions that are stored in the memory 92. The monitor 24 may be configured to communicate with the acoustic sensor 26, the ventilator 22, and/or any other components of the airway management system 20 via any suitable communication protocols, such as via wired connections, WI-FI®, or BLUETOOTH®.

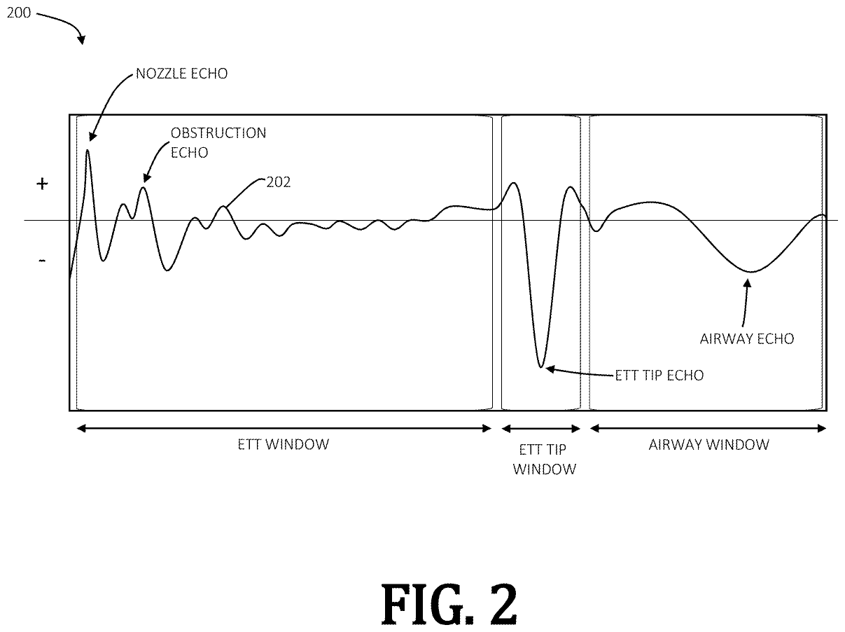

FIG. 2 depicts an example echo signal plot 200 with an echo signal 202. The echo signal 202 is generated from acoustic echoes detected by the acoustic sensor arising from acoustic pulses emitted from the acoustic sensor. In some examples, the echo signal 202 may be displayed on the monitor.

In the plot 200, the pressure amplitude is represented on Y-axis and the time delay is represented on X-axis. Deflections in the signal are indicative of reflections from cross-sectional area changes of the tracheal tube and/or the airways of the patient. Echoes that are positive indicate a cross-sectional area decrease (e.g., narrowing of the tubing or passageway), while echoes that are negative indicate a cross-sectional area increase (e.g., widening of the tubing or passageway).

Several of the echoes in the plot 200 have been labeled. A first echo is a nozzle echo that occurs from acoustic reflections at the nozzle of the acoustic sensor. The first echo is a positive deflection (positive pressure) indicating a cross-sectional area decrease. This corresponds to the decrease in the nozzle's diameter from 9 mm to 8 mm, for example. The second echo is an echo from an obstruction in the ETT, and the second echo is a positive deflection immediately followed by a negative deflection, indicating a cross-sectional area decrease and then an increase. This obstruction echo, for example, may be from a small obstruction in the ETT, from a kink in the ETT, or from a patient biting on the ETT. The amplitude of the reflection is indicative in the change in cross-sectional area. For instance, a larger obstruction would result in a larger amplitude of the echo signal. The present technology may estimate the obstruction size from the echo amplitude and the obstruction location from the echo delay time.

The third echo is an ETT tip echo that results from a reflection at the tip of the ETT. The ETT tip echo is a negative deflection indicating a cross-sectional area increase. This ETT tip echo is analyzed by the present technology to estimate the passageway size (or effective diameter or cross-sectional area) around the ETT, which corresponds to the size of the trachea around the ETT when the ETT is properly positioned in the trachea. The negative deflection echo indicates that the ETT is located in a passageway that has a larger cross-sectional area than the ETT. This would be the case for an ETT that is in a non-collapsed trachea. If this echo were to change to a positive deflection, it would indicate that the ETT is located in a passageway that has a smaller cross-sectional area than the ETT, such as in a collapsed trachea or from an obstruction at the tip of the ETT, which may be from mucus.

The last echo, referred to as the airway echo, arises from the region in the lower airways where the total cross-sectional area of the individual branching segments grows rapidly with each airway branching generation. The acoustic reflectometry system tracks the time delay of this airway echo, estimating relative changes in the distance between the ETT tip and the airway echo region. For example, if the time delay between the ETT tip echo and the airway echo is decreasing (airway echo moving to the left), then this indicates that the ETT tip is getting closer to the airway echo region or that the ETT is migrating down the trachea.

With the present technology, the focus is primarily on the ETT tip echo and changes to the ETT tip echo over time. For instance, if the ETT tip echo changes from negative to positive, such a change may be indicative of a tracheal obstruction due to tracheal collapse or potentially a mucus obstruction. An analysis of how the ETT tip echo changes over time may be useful in identifying or classifying the type of tracheal obstruction as either a tracheal collapse or some other type of obstruction, such as a mucus-based obstruction. The analysis of how the ETT tip echo changes over time may also indicate different physical properties of the collapsed trachea, such as compliance of the trachea, among other types of physical properties.

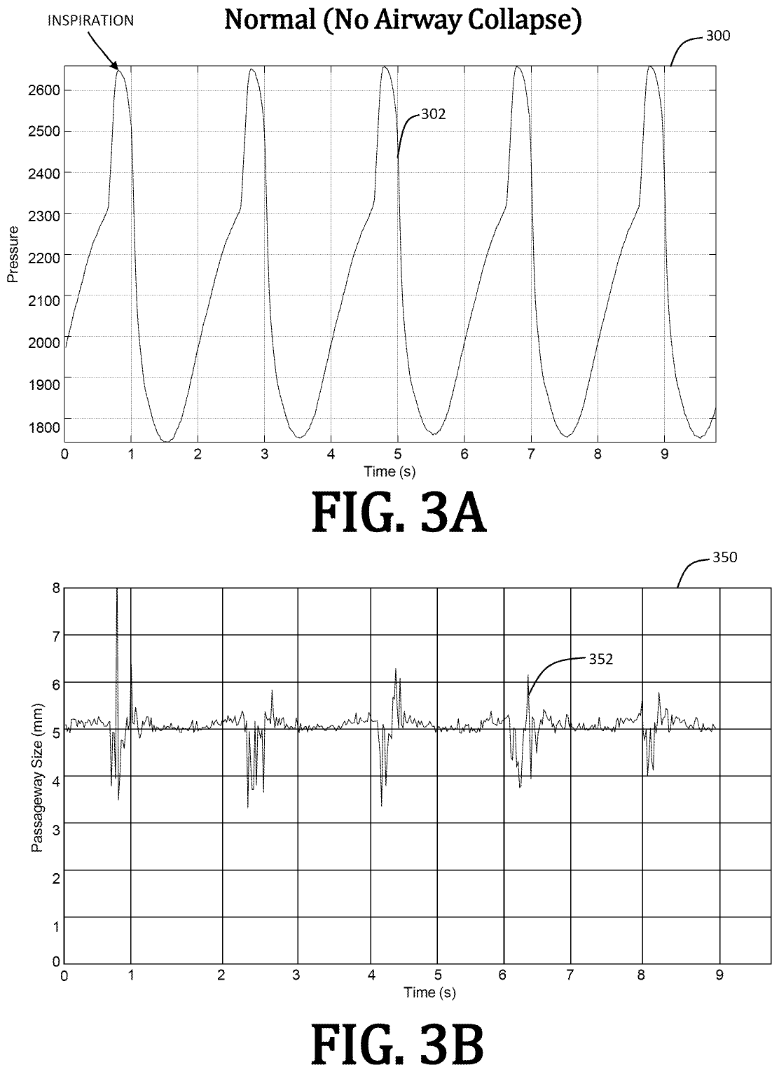

FIG. 3A depicts a pressure plot 300 of gas pressure versus time for a normal ventilation period where no airway collapse is occurring. The pressure plot 300 includes a gas pressure signal 302 over a ten second period. The gas pressure signal 302 is generated from pressure measurements taken from the acoustic sensor. As discussed above, the acoustic sensor generates acoustic pulses and detects the echoes from reflections of those acoustic pulses. The acoustic sensor may also be able to measure relative pressure changes by using a low-pass or band-pass filter such that a low-frequency pressure measurement is measured by the pressure-sensing devices (e.g., microphones, piezo-based films, etc.) of the pressure sensor. For instance, the upper frequency limit of the filter may be 100 Hz. Such a low-frequency measurement is indicative of the changes in gas pressure provided by the ventilator. Thus, inspiratory phases and expiratory phases of the breath delivery may be identified from the pressure plot 300. For example, the peaks in the pressure signal 302 correspond to inspiration of the breath cycle (e.g., when gas is being delivered by the ventilator), and the valleys in the pressure signal 302 correspond to expiration phases of the breath cycle. Of note, because the pressure measurements are relative, the units of the Y-axis of the pressure plot 300 are not absolute units of pressure but are rather analog-to-digital output values from the acoustic sensor.

FIG. 3B depicts a passageway size plot 350 of passageway size versus time for the same normal ventilation period of FIG. 3A. The passageway size plot 350 includes a passageway signal 352 that indicates the passageway size of the passageway around the tip of the ETT. The time periods and scales of pressure plot 300 and pressure plot 350 are the same, and the plots are aligned such that the effect of gas pressure changes on the passageway-size signal 352 can be seen.

The passageway-size signal 352 corresponds to passageway sizes calculated based on the ETT tip echo discussed above. In the present example, the passageway-size signal 352 is generated based on 20 millisecond (ms) measurements of the ETT tip echoes. For instance, every 20 ms, an acoustic pulse is emitted from the acoustic sensor and the corresponding ETT tip echo is detected. Such an emission and detection event may be referred to an epoch, and a passageway size may be calculated for each epoch. The frequency of 20 ms is used merely by way of example and other epoch frequencies may be used. As an example, an epoch frequency between 5-200 ms may be suitable. The passageway-size signal 352 was generated for an ETT with a 3 mm inner diameter properly placed into the trachea of a patient.

As can be seen from the passageway-size plot 350, the passageway size of the trachea remains at about 5 mm. A 5 mm passageway size for an ETT inner diameter of 3 mm is generally considered to be a good fit. The noise in the passageway-size signal 352 is primarily from corruption of the corresponding pulse echo epoch due to flow turbulence noise during breath delivery by the ventilator rather than actual changes in passageway size. For example, the periods of significant noise in the passageway-size signal 352 correspond directly to inspiration and expiration periods that can be seen in the gas pressure signal 302 of the pressure plot 350 in FIG. 3A.

Because of the noise introduced by the gas delivery process, the passageway size that is output on the monitor is often based on an averaged or otherwise smoothed analysis of the passageway-size measurements that form the passageway-size signal 352. For example, a moving average, least-squares regression, or other types of smoothing or filtering techniques may be used to provide a more consistent output of the trachea size on the monitor. Such averaging, however, may miss or overlook brief collapses or obstructions in the trachea. In addition, such averaging or filtering of the data precludes the possibility for determining other dynamic characteristics of a tracheal collapse or obstruction when such an event occurs. Thus, the fine passageway size data of passageway-size signal 352 may be retained by the present technology to provide the functionality and operations described herein.

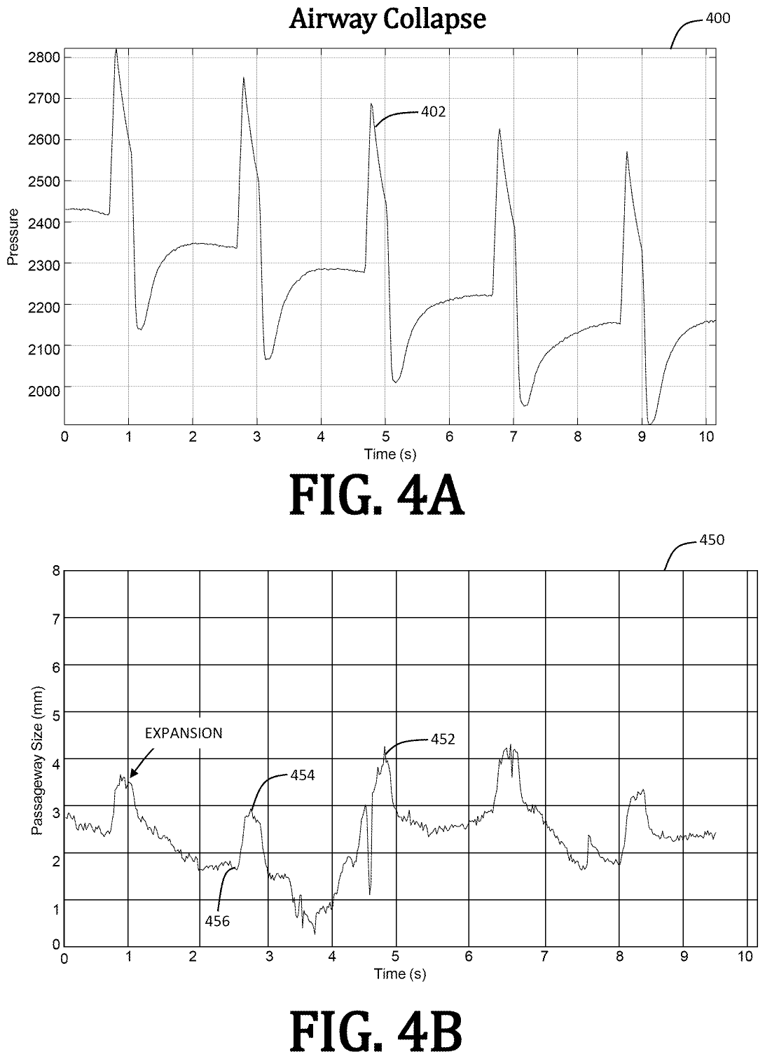

FIG. 4A depicts a pressure plot 400 of gas pressure versus time for a time period where an airway collapse (e.g., tracheal collapse) is occurring. The pressure plot 400 includes a gas pressure signal 302 over a ten second period. The pressure plot 400 is substantially similar to the pressure plot 300 of FIG. 3A but for a different time period. For instance, the gas pressure signal 402 is generated from pressure measurements taken from the acoustic sensor in the same manner as discussed above with reference to FIG. 3A. As with the pressure plot 300, inspiration and expiration phases may also be identified from the pressure plot 400. Of note, the continuous overall downward trend in pressure in plot 400 is likely due to relative baseline wander of the acoustic sensor rather than an actual decrease in delivered pressures by the ventilator.

FIG. 4B depicts a passageway size plot 450 of passageway size versus time for the same collapsed-airway ventilation period of FIG. 4A. The passageway size plot 450 includes a passageway-size signal 452 that indicates the passageway size of the passageway around the tip of the ETT (e.g., the inner diameter of the trachea). The time periods and scales of pressure plot 400 and pressure plot 450 are the same, and the plots are aligned such that the effect of gas pressure changes on the passageway-size signal 452 can be seen. The passageway-size signal 452 in passageway size plot 450 was generated in substantially the same manner as passageway-size signal 352 in plot passageway size plot 350. For instance, the passageway-size signal 452 is made up of individual passageway size calculations based on 20 ms epochs. The passageway-size signal 452 was also generated for the same patient and same ETT having an inner diameter of 3 mm.

As can be seen from the passageway size plot 450, during the tracheal collapse, the passageway size (e.g., inner trachea diameter) drops below the 3.0 mm inner diameter size of the ETT. When the passageway size drops below the inner diameter size of the ETT, the amount of breathing gases that may be delivered to the patient becomes limited and may lead to reductions in oxygen saturation of the patient, among other negative effects. Accordingly, when a passageway size drops below the inner diameter of the ETT for a threshold duration of time, an airway-collapse alarm may be activated on the monitor and/or the ventilator. Such a threshold duration of time may be between 0.2 to 1 second, such as 0.5-0.7 seconds. In other examples, the threshold duration may be longer, such as between 1-3 seconds.

From the fine measurement data that forms the passageway-size signal 452, dynamic characteristics about the tracheal collapse may also be determined or identified. For example, during in inspiration phase of a breath cycle, the trachea expands due to the pressure increase from the delivered breathing gases. The first expansion event is labeled in the passageway size plot 450, and additional expansion events can be seen at each subsequent inspiration phases. Some expansion events, such as the first expansion event and the expansion events at approximately 4.8 seconds and 6.5 seconds, may result in the trachea opening to a diameter that is larger than the inner diameter of the tracheal tube (e.g., 3 mm). Other expansion events, however, such as the second expansion event at approximately 2.8 seconds, do not result in the trachea opening to a diameter that is greater than the inner diameter of the tracheal tube.

The amount of expansion that occurs with a given applied gas pressure may be indicative of the compliance of the trachea. Tracheal collapse due to tracheomalacia may be due to the cartilage of the trachea to become soft and weak such that the trachea naturally collapses due to minimal forces being applied. In such cases, the trachea may be fairly compliant (e.g., weak or floppy). In other cases, the trachea may collapse due to muscle pressures generated from the patient (such as the patient contracting the neck muscles). In those cases, the trachea is less compliant (e.g., more rigid).

A dynamic characteristic of the compliance of the trachea may be generated from an analysis of the passageway signal 352 during and around the expansion events. For instance, a peak-to-baseline value of the passageway size may be generated. A passageway size at the peak 454 of an expansion event (e.g., during end-inspiration of a breath cycle) may be calculated as well as a passageway size baseline 456 prior to the expansion event (e.g., during end-expiration of the prior breath cycle). The peak 454 value may be an average or a maximum value of the passageway size during end-inspiration, and the baseline 456 value may be an average or a minimum value of the passageway size during the prior end-expiration. In other examples, the baseline value may be based on end-expiration of the same breath as the end-inspiration causing the expansion event.

The peak-to-baseline value may be represented as a difference between the peak value and the baseline value or a ratio between the peak value and the baseline value among other possible representations of the baseline value and the peak value. A larger difference between the peak value and the baseline value may be indicative of a greater compliance of the trachea (e.g., less rigid). For example, if the pressure of the gases from ventilation cause the trachea to expand by a greater amount, the trachea is likely more compliant. In contrast, if the pressure of the gases from ventilation cause the trachea to expand by a smaller amount, the trachea is likely less compliant (e.g., more rigid). The indications of compliance may be useful in determining treatment steps by a physician, such as whether a stent may need to be inserted into the trachea to add rigidity to the trachea. The indications of compliance may also be used in determining changes in ventilation that may help alleviate the tracheal collapse condition. For instance, for a more compliant trachea, an increase in positive end-expiratory pressure (PEEP) may help retain the trachea in an expanded state even during expiration.

In some examples, the pressure of the delivered breathing may also be used in generating an airway-compliance indicator or a tracheal-compliance indicator. For instance, while the tracheal-compliance indicator may be based on the peak-to-baseline value of the passageway signal 352, the tracheal-compliance indicator may be further based on a pressure of the delivered breathing gases. The value for the pressure of the delivered breathing gases may be obtained from sensors or settings of the ventilator or may be obtained from the pressure plot 400 and the pressure measurements made by the acoustic sensor. While the pressure measurements from the acoustic sensor may not be absolute pressure measurements, the relative pressure differences between end-inspiration and end-expiration may be determined from the pressure plot 400. For instance, a pressure difference between the peak end-inspiration pressure and the minimum end-expiration pressure may be determined and used as a factor in generating a tracheal-compliance indicator. The tracheal compliance indicator may be calculated by dividing the peak-to-baseline value from the passageway signal 352 by the pressure difference between end-inspiration and end-expiration pressures from the pressure measurements 400 obtained by the acoustic sensor or provided to the ventilator.

A graph showing the dynamic compliance of the collapsed airway around the ETT tip as a function of time may be calculated or otherwise generated by dividing the derivative of the passageway size-time signal by the derivative of the pressure-time signal obtained from the ventilator or the sensor (e.g., cm/cmH2O). The delta time value used to obtain the derivative may be the same as the sample period of 20 ms described above or it can be longer to better represent the changing passageway sizes and pressures. This estimated dynamic compliance may be used to determine the cause of the obstructive event as described above.

Trends in tracheal compliance may also be determined and used as a dynamic characteristic of the tracheal collapse. The trend may be a change over time of the tracheal-compliance indicator and/or the peak-to-baseline value. The tracheal-compliance trend may be an indication as to whether the trachea is becoming more rigid or less rigid over time or during the tracheal collapse event.

Other dynamic characteristics of the tracheal collapse may also be determined from analysis of the passageway size plot 450. For instance, a baseline trend of the passageway-size signal 452 may be determined. The baseline trend may be based on a trend of the end-expiratory baseline value during each of the breaths for the period analyzed. The baseline trend provides an indication as to whether the trachea is experiencing further overall collapse (e.g., the tracheal collapse condition is worsening). The baseline trend may be calculated over series of breaths or on a breath to breath basis.

Another dynamic characteristic of the tracheal collapse is a baseline shift. The baseline shift is a measure of how much and/or how quickly the trachea collapses during expiration. The baseline shift may be calculated based on a starting passageway size at end-inspiration (or shortly thereafter) and a passageway size at end-expiration. The baseline shift may also be based on a line fit to the passageway size during the expiration phase. A large or rapid decrease in passageway size may be indicative of a stronger force on the trachea causing the collapse. Accordingly, the baseline shift may be used to generate an airway-collapse force indicator or a tracheal-collapse force indicator.

The time duration and/or number of breaths that the passageway size remains below the ETT inner diameter size may also be calculated or tracked. In some cases, the tracheal collapse may be a brief event that resolves within a few events, and in other cases the tracheal collapse may be a more extended event.

In addition to characterizing a tracheal collapse event, the dynamic characteristics of the passageway-size signal 452 may also be used to classify an obstructive event. For example, obstructive events may be due to tracheal collapse or another type of obstruction, such as a mucus plug. A determination as to whether the obstructive event was or is a tracheal collapse or a mucus plug, for instance, may be made based on the dynamic characteristics of the passageway-size signal 452 or the calculated dynamic compliance signal described above.

As an example, the peak-to-baseline value is different for a tracheal collapse event versus a mucus plug obstruction. In the event of a mucus plug event, if the pressure generated by the ventilator does not clear the plug, the peak-to-baseline value or the calculated dynamic compliance value will be very small or zero. Alternatively, if the pressure generated by the ventilator does clear the plug then the peak-to-baseline value or the calculated dynamic compliance value will be relatively large. In contrast, for the tracheal collapse event, the peak-to-baseline value or the calculated dynamic compliance value may be within a range between greater than zero and the value indicative of a relatively large compliance following a cleared plug. The baseline shift may also be indicative of a tracheal collapse versus a mucus plug obstruction. For instance, the mucus plug may not continuously reduce passageway size during expiration, whereas a tracheal collapse may continuously reduce passageway size as the trachea further collapses over time.

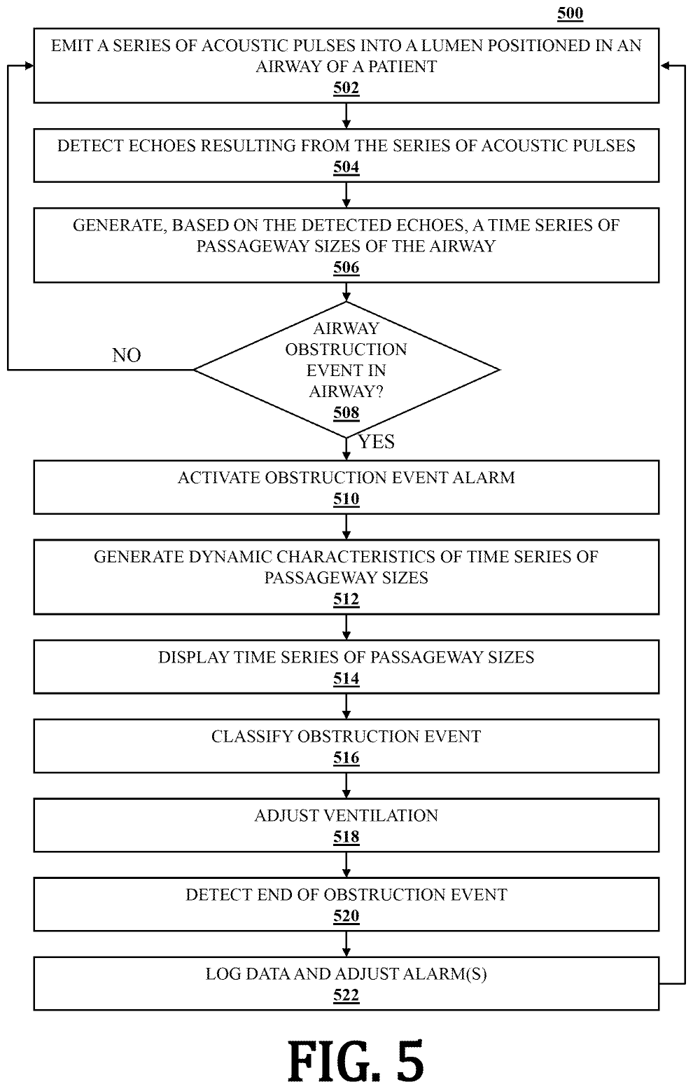

FIG. 5 depicts an example method 500 for detecting and/or identifying an airway collapse. The example method 500 may be performed by one or more of the components of the systems described herein. For example, memory or memories of devices of the system may store instructions that, when executed by one or more processors of the system, cause the system, or components thereof, to perform the operations set forth in method 500 and also described throughout the present disclosure.

At operation 502, a series of acoustic pulses are emitted into a lumen that is positioned in an airway of a patient. For example, the acoustic pulses are emitted into a tracheal tube that is positioned in the trachea of the patient. The series of acoustic pulses are emitted by the acoustic generator of the acoustic sensor. The series of acoustic pulses may be emitted at a set frequency, such as one pulse every 20 ms. As discussed above, such emission or epoch frequency may be different for different implementations or examples.

At operation 504, echoes resulting from the series of incident acoustic pulses are detected. As discussed above, the incident acoustic pulses reflect from various components and positions of the tracheal tube and the airways of the patient. Those reflected acoustics pulses (e.g., echoes) are detected in operation 504. While multiple echoes may be detected and utilized in the present technology, the primary echo of interest is the tube tip echo that occurs from the acoustic pulse reflection at the tip of the lumen (e.g., tracheal tube) in the airway of the patient. The echoes are detected by the acoustic receiver of the acoustic sensor.

At operation 506, a time series of passageway sizes of the airway at the tip of the lumen (e.g., tracheal tube) is generated based on the echoes detected at operation 504. For instance, a passageway size may be generated for each echo that is detected (e.g., a passageway size is calculated for each epoch). As discussed above, the passageway size may be generated based on the amplitude of the echo as well as whether the echo was positive or negative. For instance, a large, positive echo indicates a narrow passageway size that is smaller than the inner diameter of the lumen (e.g., tracheal tube). Conversely, a large, negative echo indicates a large passageway size that is larger than the inner diameter of the lumen. The time series of passageway sizes may be represented as the passageway-size signals discussed above and shown in FIGS. 3B and 4B.

At operation 508, a determination is made as to whether an airway obstruction event has occurred in the airway near the tip of the lumen. The determination may be based on the echoes detected in operation 504 and/or on the time series of passageway sizes of the airway. For instance, when a passageway size drops below the inner diameter of the lumen for a threshold duration of time, an obstruction event may be determined to have occurred or be occurring. Such a threshold duration of time may be between 0.2 to 1 second, such as 0.5-0.7 seconds. In other examples, the threshold duration may be longer, such as between 1-3 seconds. The determination of passageway size relative to the inner diameter of the lumen may be based on the time series of passageway sizes, which provides for a direct comparison of passageway size to inner diameter size. In other examples, the determination of passageway size relative to the inner diameter of the lumen may be based on echo polarity. For example, if a threshold number of successive positive echoes are received (or positive echoes are received for threshold duration), an obstruction event may be determined to have occurred or be occurring.

If an airway obstruction event is not determined to have occurred or be occurring in operation 508, the method 500 flows back to operation 502 where the method 500 repeats. In some examples, the method 500 may be constantly occurring and repeating. For instance, the system may be continuously emitting acoustic pulses, detecting the corresponding echoes, and performing the operations and analyses discussed herein with each newly detected echo. As the echoes are detected and the airway sizes are determined, they may be stored temporarily in a buffer. Once the buffer is full, the oldest data is deleted or discarded when the newest data is added to the buffer. When an airway obstruction event is detected, however, the time series data for the time period of the airway obstruction event may be moved or copied to permanent storage (e.g., memory of the monitor or ventilator) for later analysis by a clinician. In other examples, all the echo data may be stored and the portions of the echo data corresponding to the detected airway obstruction event may be marked or flagged for current or later review by a clinician.

If an airway obstruction event is determined to have occurred or be occurring in operation 508, the method 500 flows to operation 510. At operation 510, an obstruction event alarm may be activated. The obstruction event alarm indicates there is an airway obstruction occurring or that has recently occurred. The obstruction event alarm may be in the form of a tracheal-collapse alarm or indicator. The obstruction event alarm may be displayed on, and/or sounded from, the monitor and/or on a display of the ventilator.

At operation 512, dynamic characteristics of the time series of passageway sizes may be generated. The dynamic characteristics may include any of the dynamic characteristics discussed herein, such as peak-to-baseline value, baseline shift, baseline trend, etc. The different dynamic characteristics may be calculated as discussed above. The dynamic characteristics may also be used to generate physiological indicators about the airway, such as physiological indicators about the trachea. The physiological indicators may include the indicators discussed herein, such as an airway-compliance indicator, an airway-compliance trend, an airway-collapse indicator, a calculated dynamic compliance indicator, etc.

At operation 514, the time series of passageway sizes generated in operation 506, or a portion thereof, may be displayed. The time series of passageway sizes may be displayed as a passageway size plot, such as passageway size plot 350 or passageway size plot 450 described above with reference to FIGS. 3B and 4B, respectively. One or more of the dynamic characteristics and/or physiological indicators generated in operation 512 may also be displayed in operation 514. For instance, the dynamic characteristics and/or physiological indicators may be displayed concurrently with the passageway size plot. In other examples, the dynamic characteristics and/or physiological indicators may be displayed separately from the time series of passageway sizes. The display of the time series of passageway sizes, the dynamic characteristics, and/or the physiological indicators may be displayed on the monitor and/or the ventilator.

In some examples, the display of the time series of passageway sizes may be provided substantially in real time. For instance, as new echoes are detected and corresponding passageway sizes are determined, the display of the time series of passageway sizes may be updated such that a substantially real-time or live display of the time series of passageway sizes is displayed. Such a real time display allows for users to watch or assess the airway status in real time as well.

In some examples, method 500 may also include generating pressures of the breathing gases flowing through the lumen. Such pressures may be measured by the acoustic receivers of the acoustic sensor using a low-pass filter, as discussed above. The measured relative pressures may be displayed in the same manner as the time series of passageway sizes. For example, the pressures of the breathing gases may be displayed as a time-series plot, such as pressure plot 300 or pressure plot 400. The displayed pressure plot may also be displayed concurrently, and aligned with, the corresponding passageway size plot, such shown in the combination of FIGS. 4A-4B.

In some examples, the time series of passageway sizes, the dynamic characteristics, the physiological indicators, and/or the pressure plots may be displayed automatically upon detection of the airway obstruction event. In other examples, such features may be displayed based on or in response to receiving a selection at the monitor and/or the ventilator. For instance, upon detection of the obstruction event, a selectable user interface element may be displayed on the monitor and/or the ventilator. Selection of that user interface element then results in display of one or more of above-discussed features. In some examples, access to the display of the one or more of the above features may be available at times even when no airway obstruction event is occurring.

At operation 516, the airway obstruction event may be classified as a particular airway obstruction type. The classification of the airway obstruction event may be based on the dynamic characteristics and/or breathing gas pressures of the breathing gases delivered to the patient. For instance, as discussed above, analysis of the dynamic characteristics can reveal whether the obstruction event is due to a tracheal collapse or some other type of obstruction event, such as a mucus plug. The classification (e.g., “Tracheal Collapse”) of the obstruction event may then be displayed on at least one of the monitor or the ventilator.

At operation 518, the ventilation being delivered to the patient may be adjusted based on the detection of the airway obstruction event, the dynamic characteristics, the physiological indicators, and/or the classification of the obstruction event, among other factors. For example, a pressure setting of the delivered breathing gases may be increased for a portion of time. The increase in pressure may be in the form of an increase in PEEP to help maintain the airway in an expanded state. The increase in pressure may be for a set period of time or continue until the tracheal collapse (or other obstruction event) has resolved or ended.

The amount of increase in pressure (or the amount of change to another ventilation parameter) may also be based on the dynamic characteristics and/or the physiological parameters. For instance, if the dynamic characteristics indicate a stronger force on the trachea causing the collapse and/or a more rigid trachea, the pressure parameter of the delivered breathing gases may need to be increased by a greater amount. Conversely, if dynamic characteristics indicate that the trachea is highly compliant, a smaller pressure increase may be implemented. The increase in pressure (or other settings changes) may also occur incrementally until an upper limit is reached or until the trachea opens to a diameter that is larger than the inner diameter of the tracheal tube.

In some examples, the ventilation settings may be adjusted automatically by the ventilator without further user input. In other examples, recommended ventilation settings may be presented on a display of the ventilator and are not implemented until an acceptance selection is received from a clinician. In yet other examples, an initial adjustment may be made automatically, but any further adjustments require user input or acceptance.

At operation 520, an end of the obstruction event is detected. In some examples, the obstruction event, such as a tracheal collapse or a mucus plug, may resolve itself or end. The end of the obstruction event may be determined based on the passageway size being greater than the inner diameter size of the lumen for a threshold period of time. The threshold period of time may be greater than the threshold period of time for determining an airway obstruction has occurred. The determination of the passageway size being greater than the inner diameter of the lumen may be based on the time series of passageway sizes and/or the polarity of the detected echoes. For example, receiving negative tube tip echoes for the threshold duration of time (e.g., or receiving a threshold number of successive negative tube tip echoes) may be indicative that the that obstruction event has concluded.

At operation 522, the data generated during method 500 may be logged or otherwise stored for later review. The logged data may also be analyzed to extract data regarding the frequency, duration, and/or severity of the airway collapses or other types of airway obstruction events. Operation 522 may also include adjusting one or more alarms based on the detection of the end of the obstruction event. For example, during the obstruction event, a patient's oxygenation level (e.g., SpO2 level) may drop to a point that causes an oxygenation alarm to activate. Once the obstruction event has ended, the patient's oxygenation level will start to rise again, but the rise of the oxygenation level may take a few minutes or more. Because the system has determined the likely cause of the reduced oxygenation (e.g., the tracheal collapse) has ended, the oxygenation alarm may be downgraded, silenced, or otherwise adjusted. Upon the end of the obstruction event being detected, if the ventilation was adjusted in operation 518, the ventilation may be adjusted back to the ventilation parameters that were being utilized prior to the adjustment due to the detection of the airway obstruction event.

Subsequent to operation 522, the method 500 returns to operation 502 where the method 500 repeats. The method 500 may continuously repeat for as long as the patient is receiving ventilation. In other examples, the method 500 may be periodically or intermittently performed to assess the characteristics of the patient's airway.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers manners for carrying out the described features and functions and interfaces, and those variations and modifications that may be made to the hardware or software firmware components described herein as would be understood by those skilled in the art now and hereafter.

In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for identifying airway collapse, the method comprising:
emitting, from an acoustic sensor, a series of acoustic pulses into a tracheal tube positioned in an airway of a patient;
detecting, by the acoustic sensor, echoes resulting from the series of acoustic pulses;
generating, based on the detected echoes, a time series of passageway sizes of the airway;
based on the time series of passageway sizes, detecting an airway collapse has occurred; and
based on detecting the airway collapse has occurred, activating an airway collapse alarm.

2. The method of claim 1, wherein the time series of passageway sizes are displayed on a monitor communicatively coupled to the acoustic sensor.

3. The method of claim 1, further comprising generating dynamic characteristics of the airway collapse based on the time series of passageway sizes.

4. The method of claim 3, wherein the dynamic characteristics include at least one of a peak-to-baseline value, a baseline shift, or a baseline trend.

5. The method of claim 4, further comprising generating an airway-compliance indicator based on at least the peak-to-baseline value.

6. The method of claim 4, further comprising displaying one or more of the dynamic characteristics on at least one of a monitor communicatively coupled to the acoustic sensor or a ventilator communicatively coupled to the acoustic sensor.

7. The method of claim 3, further comprising adjusting ventilation delivered to the patient based on one or more of the dynamic characteristics.

8. The method of claim 1, further comprising:
storing the time series of passageway sizes in a buffer; and
based on detection of the airway collapse, storing the time series of passageway sizes in permanent memory.

9. A system for detecting airway collapse, the system comprising:
an acoustic sensor comprising an acoustic receiver and an acoustic generator;
a monitor communicatively coupled to the acoustic sensor;
a processor; and
memory storing instructions that, when executed by the processor, cause the system to perform a set of operations comprising:
emitting, from the acoustic generator, a series of acoustic pulses into a tracheal tube positioned in an airway of a patient;
detecting, by the acoustic receiver, echoes resulting from the series of acoustic pulses reflecting from a tip of the tracheal tube;
generating, based on the detected echoes, a time series of passageway sizes of the airway;
based on at least one of the time series of passageway sizes or the detected echoes, detecting an airway collapse has occurred; and
displaying the time series of passageway size on the monitor.

10. The system of claim 9, wherein the system further comprises a ventilator, and the monitor is integrated into the ventilator or is a stand-alone monitor separate from the ventilator.

11. The system of claim 10, wherein the operations further comprise generating dynamic characteristics of the airway collapse based on the time series of passageway sizes, wherein the dynamic characteristics include at least one of a peak-to-baseline value, a baseline shift, or a baseline trend.

12. The system of claim 11, wherein the operations further comprise, based on at least one of the dynamic characteristics, adjusting, by the ventilator, a pressure of delivered breathing gases to the patient.

13. The system of claim 9, wherein the operations further comprise, based on detecting the airway collapse has occurred, activating an airway-collapse alarm on the monitor.

14. A system for identifying an airway obstruction event, the system comprising:
a ventilator;
a monitor;
an acoustic sensor communicatively coupled to at least one of the monitor or the ventilator, the acoustic sensor comprising an acoustic receiver and an acoustic generator;
a processor; and
memory storing instructions that, when executed by the processor, cause the system to perform a set of operations comprising:
emitting, from the acoustic generator, a series of acoustic pulses into a tracheal tube positioned in an airway of a patient;
detecting, by the acoustic receiver, echoes resulting from the series of acoustic pulses reflecting from a tip of the tracheal tube;
generating, based on the detected echoes, a time series of passageway sizes of the airway;
based on at least one of the time series of passageway sizes or the detected echoes, detecting an airway obstruction event has occurred;
generating dynamic characteristics for the time series of passageway sizes of the airway; and
based on the dynamic characteristics, classifying the airway obstruction event as an airway collapse.

15. The system of claim 14, wherein the dynamic characteristics include at least one of a peak-to-baseline value, a baseline shift, or a baseline trend.

16. The system of claim 14, wherein the dynamic characteristics include a peak-to-baseline value, wherein the peak-to-baseline value is calculated based on (1) a peak passageway size at end-inspiration of a breath delivered by the ventilator and (2) a baseline passageway size at end-expiration of a breath delivered by the ventilator.

17. The system of claim 14, wherein the operations further comprise, based on detecting the airway obstruction event, adjusting, by the ventilator, ventilation being delivered to the patient.

18. The system of claim 17, wherein adjusting the ventilation includes increasing a positive end-expiratory pressure (PEEP) setting.

19. The system of claim 17, wherein adjusting the ventilation is further based on the dynamic characteristics.

20. The system of claim 14, wherein the operations further comprise:
based on the time series of passageway sizes, detecting an end to the airway obstruction event; and
downgrading an oxygenation alarm based on detecting the end to the airway obstruction event.
